(19) United States
(12) Patent Application Publication
(10) Pub. No.: US 2026/0015578 A1
(43) Pub. Date: Jan. 15, 2026

(54) PERFUSABLE SCAFFOLDS FOR 3D CELL CULTURES AND METHODS OF USE

(71) Applicant: The Trustees of Indiana University, Bloomington, IN (US)

(72) Inventors: Feng GUO, Bloomington, IN (US); Hongwei CAI, Bloomington, IN (US)

(21) Appl. No.: 19/252,737

(22) Filed: Jun. 27, 2025

Related U.S. Application Data
(60) Provisional application No. 63/665,708, filed on Jun. 28, 2024.

(51) Int. Cl. C12N 5/00; C12N 5/0793

(52) U.S. Cl. CPC C12N 5/0068; C12N 5/0062; C12N 5/0619; C12N 2506/45; C12N 2513/00; C12N 2539/00

(57) ABSTRACT

Disclosed herein is a perfusable scaffold for used in a three-dimensional cell culture and method of fabrication of a perfusable three-dimensional cell culture containing a perfusion scaffold. Also disclosed herein are the perfusable three-dimensional cell cultures and uses thereof.

### CROSS REFERENCE TO RELATED APPLICATIONS

This application claims the benefit under 35 U.S.C. § 119 (e) of U.S. Provisional Application No. 63/665,708 entitled “DEVICE AND METHODS FOR ENGINEERING PERFUSABLE 3D CELL CULTURES”, filed on Jun. 28, 2024, which is incorporated by reference in its entirety.

### FIELD OF THE INVENTION

The present disclosure relates generally to a perfusable scaffold for generation of three-dimensional cell cultures such as organoids. The present disclosure also relates to methods for constructing three-dimensional cell cultures with an in vivo like perfusion system and uses thereof.

### BACKGROUND

Three-dimensional cell cultures, such as organoids derived from pluripotent stem cells, represent a cutting-edge human-based model that closely recapitulates the cellular composition and the complex self-organizing properties of an early human brain. These cell cultures, such as brain and neural organoids, provide a promising platform for studying neurodevelopmental processes, disease modelling, and drug testing by capturing multiple histological and functional aspects of the human brain. Despite their advanced capabilities, one of the main limitations of current organoid technology is the absence of an in vivo-like tubular perfusion system within the cell culture. In a natural in vivo setting, cells are positioned only a few hundred microns away from the nearest capillaries, ensuring adequate nutrient and oxygen supply essential for cellular activities and growth. However, in current organoid models, cells are self-organized into a spherical formation, often resulting in several millimeters from the nearest perfusion surface. This significantly limits efficient nutrient and waste exchange, ultimately restricting the growth, maturation, and overall utility of the cell culture.

The present disclosure describes devices and methods for constructing perfusable three-dimensional cell cultures with an in vivo like tubular perfusion system to overcome the challenges described above.

### SUMMARY OF THE INVENTION

A first aspect of the invention includes a perfusable scaffold comprising at least one layer of interconnected perfusable channels wherein each perfusable channel contains at least one opening on the exterior surface of the perfusable channel.

A second aspect of the invention includes a perfusable three-dimensional cell culture wherein a perfusable scaffold is positioned within the cell culture.

A third aspect of the invention includes a method for constructing a perfusable three-dimensional cell culture.

In a first embodiment is a perfusable scaffold for perfusion of substances within a cell culture, comprising at least one layer of perfusable channels wherein each perfusable channel of the scaffold contains a hollow path extending the length of the channel and a plurality of openings through the exterior surface of the channel and wherein each perfusable channel of the scaffold is connected to at least one other perfusable channel of the scaffold.

In a second embodiment, the perfusable scaffold is composed of a biocompatible material.

In a third embodiment, the hollow path is about 7 micrometers to about 10 millimeters in diameter.

In a fourth embodiment, the openings in the perfusable channels have average diameters from about 1 nm to about 500 μm.

In a fifth embodiment, the perfusable channels are oriented in a grid arrangement.

In a sixth embodiment, the perfusable channels are oriented in a branched arrangement.

In a seventh embodiment, the scaffold is configured to fit within a microreactor, a culture dish, a culture flask, a culture plate, or a culture well plate.

In an eight embodiment, a perfusable three-dimensional cell cultures comprising a cell culture and the perfusable scaffold, wherein the perfusable scaffold is positioned within the cell culture.

In a ninth embodiment, the three-dimensional cell culture is an organoid.

In a tenth embodiment, the organoid is a midbrain organoid.

In an eleventh embodiment, the average distance from a cell of the cell culture to the surface of a perfusable channel is about 10 micrometers to about 5 millimeters.

In a twelfth embodiment, liquid or gas is perfused through the openings on the surface of the perfusable channels of the scaffold into the cell culture.

In a thirteenth embodiment, a method for constructing a perfusable three-dimensional cell culture, comprising the steps of a) inducing pluripotent stem cells to begin differentiation; b) embedding the differentiating cells onto at least one layer of perfusable scaffold about three days after induction; c) culturing the cells to expand across the external surfaces of the perfusable scaffold; and d) perfusing liquid or gas through the perfusable scaffold into an area around the cells of the three-dimensional cell culture.

In a fourteenth embodiment, the pluripotent stem cells are induced to differentiate into neural cells.

In a fifteenth embodiment, the liquid perfused through the scaffold comprises one or more of cell nutrients, salts, or growth factors.

In a sixteenth embodiment, the liquid perfused through the scaffold comprises one or more of active agent in the form of a small molecule, protein, antibody, virus, cell, viral, or nucleic acid.

In a seventeenth embodiment, the active agent is a therapeutic agent.

### DESCRIPTION OF THE FIGURES

The features of the present disclosure according to some examples or embodiments will now be described, purely by way of example, with reference to the accompanying drawings, in which:

FIG. 1A is a schematic representation of perfusable midbrain-like organoid engineering and culture timeline.

FIG. 1B is a representative bright-field image of a perfusable organoid captured at 2 months (from EB formation).

FIG. 1C and FIG. 1D are representative hypoxia staining images of perfusable organoids and spherical organoids at 2 months using a pimonidazole-based hypoxia probe. Hypoxic cells are in red and DAPI in gray. Scale bars, 200 μm.

FIG. 1E are schematic representations showing the design principles of perfusable organoids. The engineering method enhances the perfusion of oxygen and molecules by reducing the Dnps (distance to the nearest perfusable surface) as in vivo perfusion network.

FIG. 1F are Violin plots showing representative quantification results of Dnps in human cortex slide (in vivo), spherical organoid, and perfusable organoid at 2 months. Each circle represents the Dnps of a cell.

FIG. 2A shows representative immunostaining images for progenitor maker SOX2, mature neuron marker MAP2, and apoptosis marker cleaved caspase-3 (Cas3) at days 10, 30, and 60. Scale bars=200 μm.

FIG. 2B is a graphical representation of the quantification of the percentage of progenitor (SOX2+) cells in the whole organoid at days 10, 30, and 60. (mean±SEM, n=6 from 3 independent experiments).

FIG. 2C is a graphical representation of the quantification of the percentage of apoptotic dead (Cas3+) cells in the whole organoid at days 10, 30, and 60. (mean±SEM, n=6 from 3 independent experiments)

FIG. 3A are schematics of perfusable and spherical human midbrain organoids on the MEA plates showing the superior perfusion of perfusable organoids reduced the low-activity center caused by necrosis and enhanced the diffusion for compound screening.

FIG. 3B are representative electrophysiological measurements of 3-month perfusable and spherical organoids. The blue color shows the smoothed activity heat map of organoids. The network plot showing the functional connectivity is overlayed on the heatmap. Line thickness and color represent the strength of correlation between electrodes in the network, and node size represents the connection numbers per electrode. Functional connectivity was determined by analyzing the spike time tiling coefficient from spontaneous activity.

FIG. 3C is a graphic of quantified network indexes (density, global efficiency, local efficiency, and average clustering) of 3-month perfusable and spherical organoids (mean±s.e.m., n=7, 3 independent experiments) showing the significantly enhanced network function in perfusable organoids.

FIG. 3D are smoothed spike activity heatmaps indicate the spontaneous activity changes of the same perfusable or spherical organoid before and 20 minutes after 50 nM fentanyl treatment.

FIG. 3E is a graphic of the mean firing rate (Hz) of 3-month perfusable and spherical organoids (mean±s.e.m., n=7, 3 independent experiments) before and after exposure to 50 nM of Fentanyl.

### DETAILED DESCRIPTION

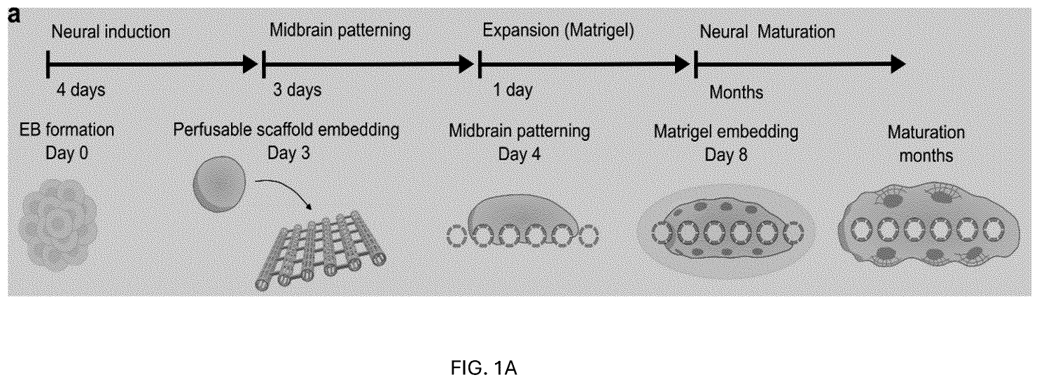

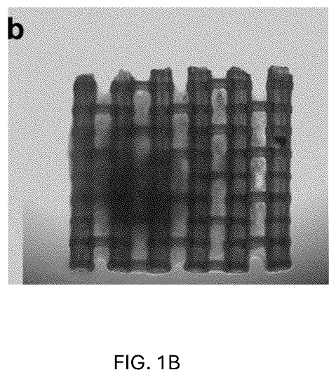

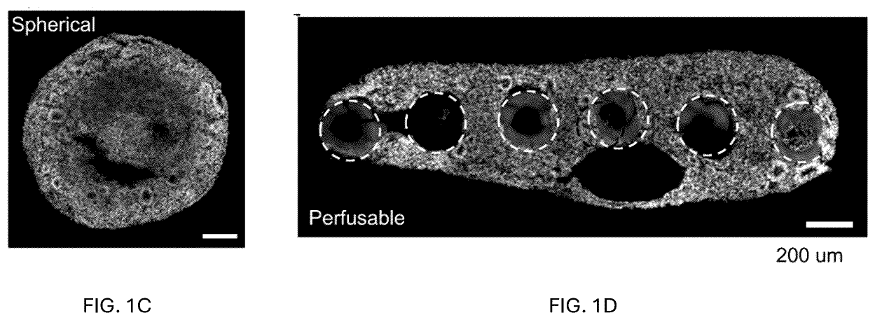

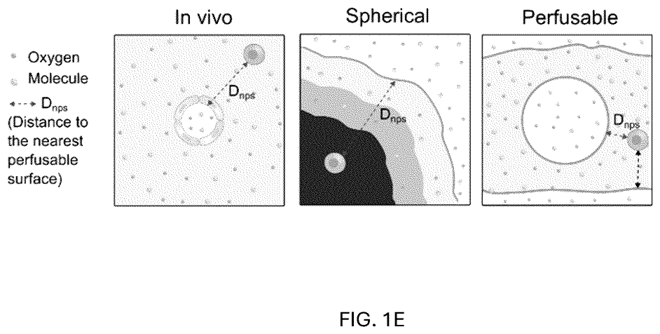

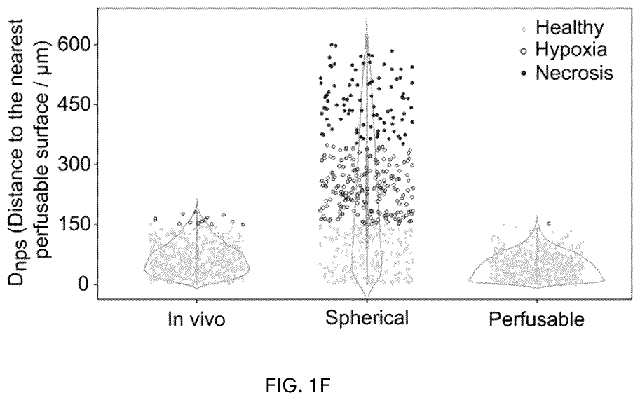

In a conventional three-dimensional cell culture, such as a spherical organoid culture, the viability and development of the cells inside the organoids are limited by the deficient supply of oxygen and nutrients via surface diffusion. To mimic the perfusable tube system of capillaries in an organ and recapitulate in vivo perfusion, Applicant incorporated a perfusable scaffold consisting of perfusable channels into the cell culture. (See FIG. 1A). The perfusable scaffold enables the nutrients and oxygen perfusion inside the cell culture, and guides the cell culture into a flattened shape, further enhancing the surface diffusion by increasing the surface-to-body ratio of the cell culture.

Additionally, perfusable three-dimensional cell cultures according to the present disclosure allow compounds and therapeutics to penetrate deeply inside the cell culture, rather than be limited to the surface of the spherical shaped culture, thereby better mimicking the in vivo pharmacokinetics. An image of an embodiment of a perfusable cell culture according to the present disclosure is show in FIG. 1B.

As used herein, the three-dimensional in vitro cell cultures refer to cell cultures that are allowed to grow and interact in three-dimensions in the cell culture environment, mimicking their natural in vivo environment more closely than traditional two-dimension cell cultures. The term “organoid” refers to one type of three-dimensional cell culture that mimic the structure and function of real organs. The terms “organoid” and “three-dimensional (3D) in vitro cell cultures” are used interchangeably herein. Organoids are generated from stem, pluripotent, induced pluripotent, or progenitor cells from human or animal origin. The stem and/or pluripotent stem cells are induced to form the cells of the specific organ of interest, such as brain, liver, or pancreas. For example, the pluripotent cells may be induced to transition into mature cells present in the brain such as brain and neural cells to form a three-dimensional cell culture mimicking the midbrain of a human.

As used herein, the term “perfusable” refers to the flow of substances such as fluids, nutrients, oxygen, chemical molecules and/or biochemical molecules through the channel networks of the scaffold. These substances are perfused from the scaffold into the three-dimensional cell culture. The perfusion of substances may occur through the movement of the tissue culture medium through the channel network into the interior of the cell culture through the normal movement and/or shaking of the tissue culture flask or plate during cell culturing. Alternatively, the substances may be perfused through the channel network at a specified rate and time through connection of the perfusable scaffold to an external microfluid perfusion system. Further, as used herein, the perfusion may also encompass the passive movement of substances from the channel networks of the scaffold into the cell culture.

### Perfusable Scaffolds

Perfusable scaffolds disclosed herein may be formed from any biocompatible material, such as glass or a biocompatible resin or plastic. Biocampatible materials for use in the perfusable scaffold include natural polymers, synthetic polymers, hydrogels, ceramics, and composites. Preferably, the scaffold is composed of a biocompatible material that can be 3D printed into a scaffold having the desired shape and dimensions.

The scaffold is composed of a series of perfusable channels. The perfusable channels may be in the shape of hollow meshed or perforated tubes. In one embodiment, the tubes are rounded in shape. However, the perfusable channels are not limited to having a rounded surface as long as they include a continuous hollow space the length of channel to allow uninterrupted flow of liquid or gas through the channel. The perfusable channels are preferably about 7 to about 200 micrometers in diameter. However, one of skill in the art would appreciate that the diameter size may be larger, including channels up to 10 mm in diameter, or smaller depending upon design choice. The ends of the perfusable channels may be open to allow the passive movement of substances through the length of the channel, such as movement of liquid through a straw. Alternatively, the ends of the perfusable channels may be closed apart from a single point of entry into a perfusable channel of the scaffold to allow substances to be perforated into and through the perfusable channel at a specified rate and timing. In this embodiment, a syringe or microfluid perfusion system may be attached to this single point of entry in the scaffold to move substances into and through the scaffold.

The surface of the perfusable channel contains a plurality of openings, which may be in the form of perforations or holes, to allow the movement of liquids or gases from the channel to the cell culture. Openings in the perfusable channels of the scaffold from which liquids or gases may perfuse or diffuse into the cell culture may have average diameters from about 1 nm to about 500 μm. In one embodiment, the scaffold was designed as parallel meshed tubes of 200 μm diameter (10 μm openings) with 300 μm inter-tube distance. The size of the perforations or holes can range from nm to mm, depending upon the volume and rate of perfusion desired and the type of substance to be perfused into the cell culture via the scaffold. The plurality of openings through the exterior of the perfusable channel create a allow the exterior of the perfusable channel to be very porous and better imitate a capillary within the cell culture.

The scaffold may be designed as a series of parallel perfusion channels that are interconnected to maintain their alignment and/or provide structural support. This scaffold design is show in the illustration of FIG. 1A. Alternatively, the scaffold may be in the form of interconnected perfusion channels wherein the connection between the perfusion channels allows for the movement of substances between the perfusion channels. In one embodiment, the scaffold may be designed in a grid patter such that the perfusion channels positioned along an x-axis are interconnected with perfusion channels along positioned along a y-axis and allowing substances to flow between all of the interconnected channels of the grid pattern. In an alternative embodiment the scaffold may be designed in a branched pattern to mimic the branching of the vascular system, particularly the branching of capillaries, such that a perfusable channel my have one or more perfusable extending (“branching”) from it allowing the flow of substances from the perfusable channel through its branches. In a further branding embodiment, the perfusion channel “branches” may be of a smaller diameter than the perfusion channel from which they extend. One of skill in the art would appreciate that the number and geometric arrangements of the perfusable channels of the scaffold may be modified depending upon the size of organoid desired and the minimum distance from cell to scaffold desired.

Considering the typical intercapillary distance is around several tens to hundreds of microns, the preferable spacing of the tubes or channels of the scaffold mimics this intercapillary distance, such that the average distance from a cell within the cell culture to the nearest perfusable channel of the scaffold is within 2 μm-5 mm. Ideally, the average distance is within 2-500 μm, 25-400 μm, 50-300 μm, 100-300 μm, or 100-200 μm. Most preferably, the average distance is 150 μm from the cell to the nearest perfusable or diffusive surface of a perfusable channel of the scaffold.

Perfusable three-dimensional cell cultures of the present disclosure include at least one layer of perfusable scaffold. Larger organoids including more than one layer of perfusable scaffold are also contemplated. Perfusable organoids may include at least 1, at least 2, at least 3, or at least 4 layers of perfusable scaffold.

The perfusable scaffold may be designed to fit within a microreactor, a standard culture dish, tissue culture flask, tissue culture plate or tissue culture well plate.

### Preparation of Perfusable Three-Dimensional Cell Cultures.

Disclosed herein is a method for constructing perfusable of diffusible three-dimensional cell cultures. Pluripotent stem cells for use in this method may be obtained from a variety of sources, including embryonic stem cells, tissue stem cells, or induced pluripotent stem cells.

Methods for preparing three-dimensional cell cultures, such as organoids, are well known in the art and may be utilized in the preparation of the perfusable three-dimensional cell cultures described herein. Exemplary methods and techniques include Cell Stem Cell, 2016 Aug. 4; 19(2):248-257, incorporated by reference herein. The preparation of the three-dimensional cell culture includes inducing the pluripotent stem cells to develop into the desired mature cell type for the culture.

The cells are then embedded onto a perfusable scaffold about three days after induction and cultured to expand across the external surfaces of the perfusable scaffold. The presence of the perfusable scaffold within the cell culture promotes the growth of cells in a flat shape along the surface of the scaffold as opposed to the growth into the rounded shape of standard three-dimensional cell cultures. This flattened shape of the culture provides additional benefits-allowing ease in measurement and imaging of the cells within the culture.

### Use of Perfusable Three-Dimensional Cell Cultures

Perfusable three-dimensional cell cultures as described herein may be used in the same manner as traditional organoids. However, the incorporation of the perfusable scaffold within the cell culture allows the health of the cells to be maintained for a longer period of time. In one embodiment described herein, Applicant has demonstrated fabrication of perfusable midbrain organoids using the three-dimensional scaffold. Compared to traditional spherical cell cultures, the perfusable midbrain organoids exhibited several enhanced characteristics: they show reduced hypoxia, sustain higher level of neurogenesis, and have greater midbrain specificity and improved network functionality Applicant demonstrated that this scaffold enhances the diffusion of molecules of various sizes, from oxygen to protein-based growth factors, overcoming the limitations of diffusion in traditional spherical organoid cultures. Enhanced perfusion of oxygen, nutrients, and growth factors not only eliminated necrosis but also promoted more specific differentiation in the midbrain region and improved neuronal network functions. (See FIG. 1E).

Additionally, perfusable three-dimensional cell cultures as described herein are superior for testing the impact of a stimulus upon the cells of the culture. The perfusable system facilitates efficient compound diffusion, broadening its potential applications in organoid-based therapeutics and drug screening. The incorporation of the perfusable network within these cell cultures significantly improved the diffusion efficiency of substances and compounds for testing. For instance, when treated with fentanyl, the perfusable cell cultures demonstrated responses that were more physiologically relevant, highlighting their potential as a model for studying drug effects in a dynamic, in vivo-like environment.

### EXAMPLES

### Example 1: Function of the Perfusable Scaffold

To validate the function of the perfusable scaffold, different-sized molecules were infused into the organoids to study the diffusion dynamics, mimicking the diffusion dynamics of a variety of oxygen, nutrients, growth factors, and compounds. With enhanced perfusion of oxygen, nutrients, and growth factors, perfusable organoids showed enhanced maturation of functional neuronal networks, characterized by increased electrical activity and functional connectivity. Moreover, the perfusable scaffolds are compatible with standard 96 well plate organoid culture protocol. To prevent necrosis and take advantage of the enhanced perfusion of growth factors, each organoid was incorporated onto perfusable scaffold at day 3 induction in 96 well plates and was then cultured following the same differentiation protocol as the conventional spheroid organoids. (FIG. 1F). Compared to the spherical organoids (FIG. 1C), the perfusable scaffold substantially eliminated the interior hypoxia core (FIG. 1D).

### Example 2: Evaluation of Necrosis and Sustained Neurogenesis in the Perfusable Organoids

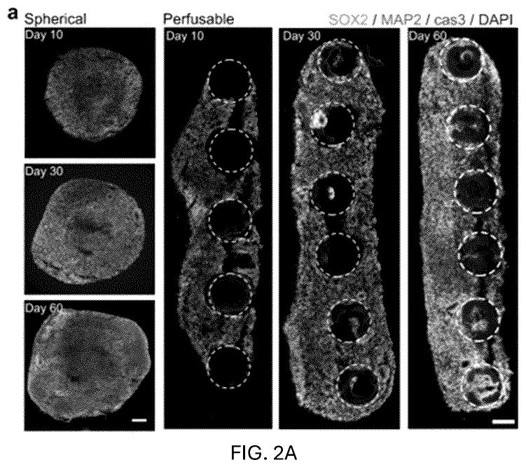

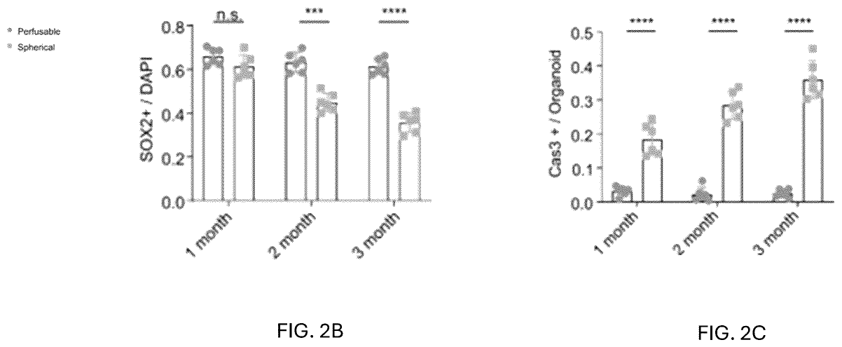

Applicants conducted a characterization of the development and necrosis within the perfusable organoids and spherical organoids. Immunostaining of organoids for progenitor marker SOX2, mature neuron marker MAP2, and apoptosis marker cleaved caspase-3 (Cas3) was conducted at days 10, 30, and 60 on perfusable and spherical organoids. As shown in FIG. 2A, the perfusable organoids showed significant reduced hypoxia and almost no necrosis, while a significant hypoxia area could be observed in the spherical organoids. Quantification of the percentage of progenitor cells (SOX2+) within the organoid is shown in FIG. 2B which the quantification of the percentage of dead cells (Cas3+) is shown in FIG. 2C.

### Example 3: Evaluation of Perfusable Organoids as an Advanced Electrophysiology Platform for Substance Testing

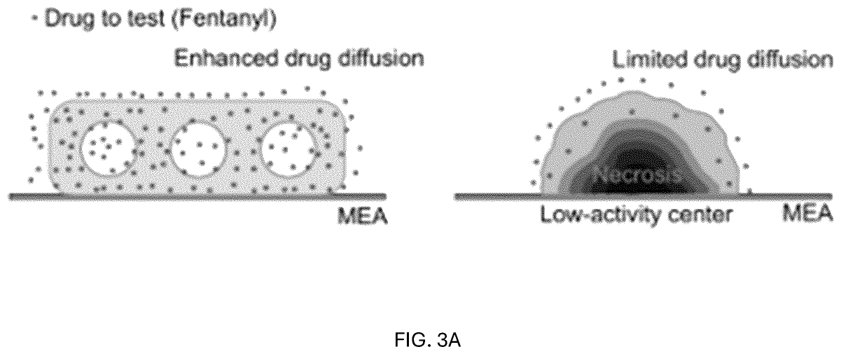

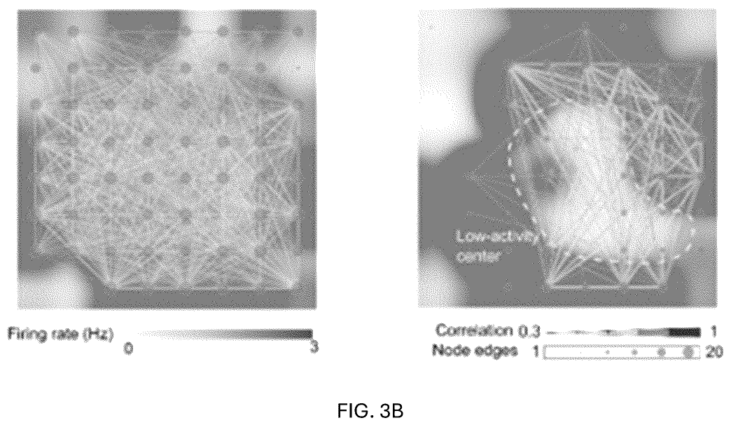

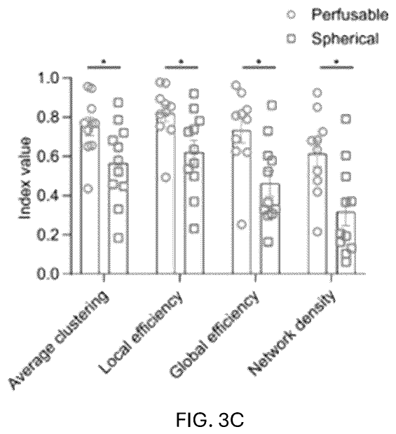

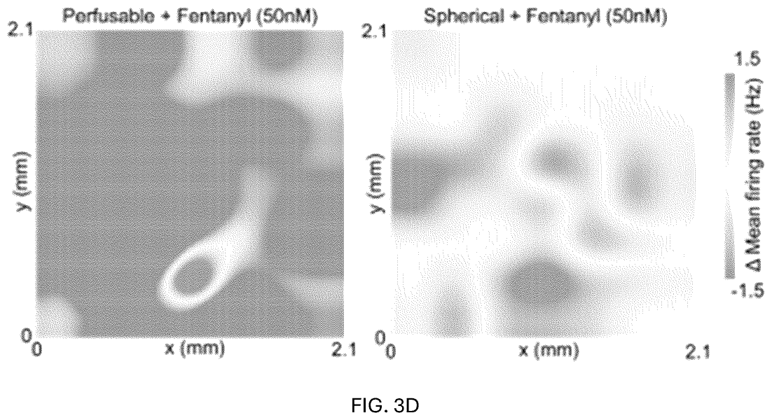

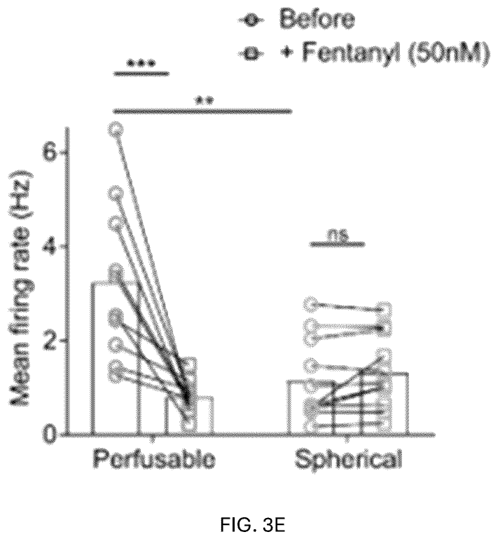

Measured by microelectrode arrays, the human brain organoids showed complex oscillations and information processing abilities as an early brain, which demonstrated the potential of organoid functional study and screening for compounds. However, conventional MEA setups can impede the perfusion of oxygen, nutrients, and test compounds by limiting organoid contact with the culture medium, leading to necrosis at the MEA surface and potentially skewing functional measurements. (FIG. 3A). Applicant hypothesized that the method disclosed herein would enhance perfusion in MEA-attached organoids, thereby improving functional network connectivity and producing more in-vivo-like responses to compounds. To test this hypothesis, Applicant performed extracellular recordings of both perfusable organoids and spherical organoids at 2 months old using MEA. Analysis of the spontaneous activity heatmaps for spherical organoids revealed a distinct low-activity center indicative of a necrotic core resulting from impaired perfusion at the MEA surface. (FIG. 3B) The functional connectivity within the spherical organoids was also impaired, only a few long connections went across the low-activity center. With the perfusable scaffold, spontaneous activity could be detected from almost all the 8×8 electrode arrays, as well as abundant functional connections among electrodes, including short-range connections among adjacent electrodes and long-range connections spanning the array. Quantified by classical network indexes (clustering, efficiency, and density), the functional network in perfusable organoids was significantly better than in the spherical organoids. (FIG. 3C) Moreover, the perfusable organoid facilitated superior compound perfusion, which could better mimic the in vivo response. We used fentanyl, a u-opioid receptor (mOR) agonist which induces strong analgesic responses in vivo, as an example. When treated with 100 nM fentanyl, the perfusable organoids generally showed decreased activity across most electrodes, aligning with expected in-vivo outcomes. (FIG. 3D and FIG. 3E) While the spherical organoids exhibited increased activity at most electrodes under similar conditions, likely due to inadequate fentanyl perfusion and a consequent reduction in inhibition from GABAergic neurons not detected by the MEA. Our engineered perfusable organoids demonstrated enhanced MEA compatibility and electrophysiological functions, and more accurate pharmacological responses, presenting a promising platform for electrophysiological studies and compound testing.

I claim:

1. A perfusable scaffold for perfusion of substances within a cell culture, comprising at least one layer of perfusable channels wherein each perfusable channel of the scaffold contains a hollow path extending the length of the channel and a plurality of openings through the exterior surface of the channel and wherein each perfusable channel of the scaffold is connected to at least one other perfusable channel of the scaffold.

2. The perfusable scaffold of claim 1 wherein the perfusable scaffold is composed of a biocompatible material.

3. The perfusable scaffold of claim 1 wherein the hollow path is about 7 micrometers to about 10 millimeters in diameter.

4. The perfusable scaffold of claim 1 wherein the openings in the perfusable channels have average diameters from about 1 nm to about 500 μm.

5. The perfusable scaffold of claim 1 wherein the perfusable channels are oriented in a grid arrangement.

6. The perfusable scaffold of claim 1 wherein the perfusable channels are oriented in a branched arrangement.

7. The perfusable scaffold of claim 1 wherein the scaffold is configured to fit within a microreactor, a culture dish, a culture flask, a culture plate, or a culture well plate.

8. Perfusable three-dimensional cell cultures comprising a cell culture and the perfusable scaffold of claim 1, wherein the perfusable scaffold is positioned within the cell culture.

9. Perfusable three-dimensional cell cultures of claim 8 wherein the three-dimensional cell culture is an organoid.

10. Perfusable three-dimensional cell cultures of claim 9 wherein the organoid is a midbrain organoid.

11. The perfusable three-dimensional cell culture of claim 8 wherein the average distance from a cell of the cell culture to the surface of a perfusable channel is about 10 micrometers to about 5 millimeters.

12. The perfusable three-dimensional cell culture of claim 8 wherein liquid or gas is perfused through the openings on the surface of the perfusable channels of the scaffold into the cell culture.

13. A method for constructing a perfusable three-dimensional cell culture, comprising the steps of
a) inducing pluripotent stem cells to begin differentiation.
b) embedding the differentiating cells onto at least one layer of perfusable scaffold about three days after induction;
c) culturing the cells to expand across the external surfaces of the perfusable scaffold;
d) perfusing liquid or gas through the perfusable scaffold into an area around the cells of the three-dimensional cell culture.

14. The method of claim 13 wherein the perfusable scaffold comprises at least one layer of perfusable channels wherein each perfusable channel of the scaffold contains a hollow path extending the length of the channel and a plurality of openings through the exterior surface of the channel and wherein each perfusable channel of the scaffold is connected to at least one other perfusable channel of the scaffold.

15. The method of claim 13 wherein the pluripotent stem cells are induced to differentiate into neural cells.

16. The method of claim 13 wherein the liquid perfused through the scaffold comprises one or more of cell nutrients, salts, or growth factors.

17. The method of claim 13 wherein the liquid perfused through the scaffold comprises one or more of active agent in the form of a small molecule, protein, antibody, virus, cell, viral, or nucleic acid.

18. The method of claim 17 wherein the active agent is a therapeutic agent.
